(19) United States
(12) Patent Application Publication
(10) Pub. No.: US 2003/0204131 A1
(43) Pub. Date: Oct. 30, 2003

(54) Medical device or system and method for the operation thereof

(75) Inventors: Stefan Aschoff, Bubenreuth (DE); Joerg Bindner, Weisendorf (DE); Roland Weidner, Zapfendorf (DE)

Correspondence Address: SCHIFF HARDIN & WAITE, 6600 SEARS TOWER, 233 S WACKER DR, CHICAGO, IL, 60606-6473, US

(73) Assignee: Siemens Audiologische Technik GmbH

(21) Appl. No.: 10/412,376

(22) Filed: Apr. 11, 2003

(30) Foreign Application Priority Data
Apr. 29, 2002 (DE) 10219097.6

(51) Int. Cl. A61B 5/00

(52) U.S. Cl. 600/300

(57) ABSTRACT

The operation of a medical device or system is simplified and improved by storing data with respect to possible processing or operating sequences in a data bank. The stored data are compared to processing or operating steps executed by a user. Dependent on the comparison result, prompts to further, meaningful processing or operating steps are generated, and settings and functions of the medical device or system are automatically implemented.

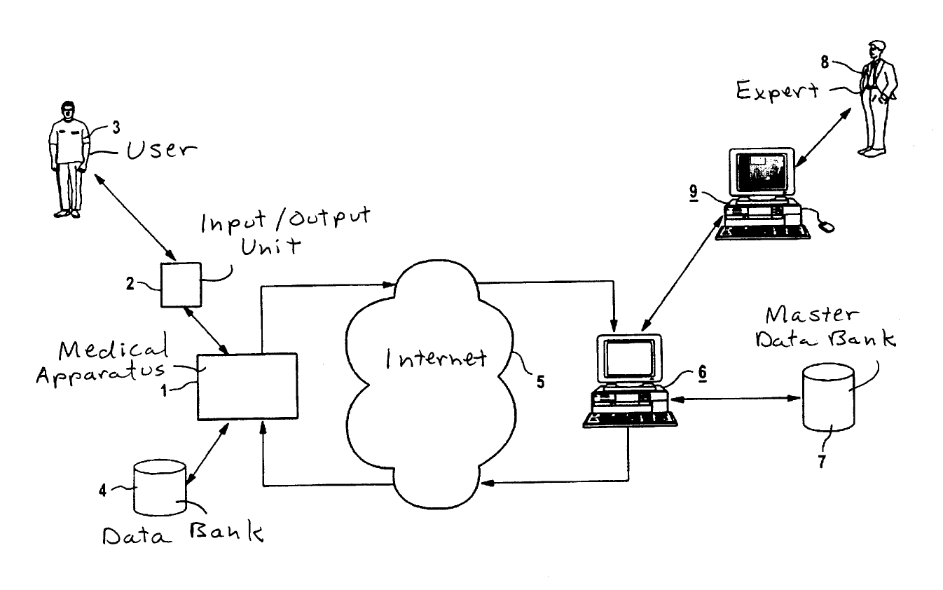

### BACKGROUND OF THE INVENTION

1. Field of the Invention

The present invention is directed to a method for the operation of a medical device or system by a person by executing a sequence of processing or operating steps. The invention is also directed to a medical device or system having an operating device for the implementation of processing or operating steps at the medical device or system.

2. Description of the Prior Art

Software-controlled medical devices as well as systems in combination with a software-controlled medical device increasingly have more functionality and use possibilities since many technical executions and functions can be rather simply realized with software. This can result in the complex operating possibilities can no longer be made transparent for a user, which increases the probability of operating errors of the devices and the appertaining software. Further, it is not easy for product managers and product developers to assess the possible sources of error potentials.

In recent developments of medical devices and systems, detailed studies, discussion groups, customer surveys and the like have been implemented in order to recognize the possible sources of error in the operation of the devices and systems. As soon as an operating software of the devices and systems is on the market, training is implemented, informational material is produced and questionnaires may be sent and evaluated, in order, among other things, to recognize sources of error and to confront them.

The medical devices and systems of the type addressed herein are complex medical devices and systems such as, for example, tomography devices, angiography devices or hearing aid devices that can be connected to a programming system.

The term “operating” is used herein in a broad sense as encompassing the control, programming, setting, maintenance, adaptation and/or monitoring of the medical device or system.

German OS 199 33 524 discloses a method for entering data into a system. In order to make the entry of data easier for users, in response to an input on the part of the user, one or more terms matching optimally well with this entry are identified as recognized terms, and a dependability value is defined for each of these recognized terms, and the terms allocated to an input are handled taking their dependability values into consideration.

### SUMMARY OF THE INVENTION

An object of the present invention is to simplify and improve the operation of medical devices and systems.

In a method for the operation of a medical device or system by a person by means of executing a sequence of processing or operating steps, this object is achieved by comparing the executed sequence of processing or operating steps to data representing possible processing or operating sequences deposited in a data bank, and optimizing the operation dependent on the result of the comparison.

In a medical device or system (medical apparatus) having an operating device for the implementation of processing or operating steps at the medical device or system, the object is achieved by a data bank connected to the medical device or system for storing data representing possible processing or operating sequences of the medical device or system, a comparator for comparing the processing or operating steps to the data stored in the data bank, and a control unit for controlling an output unit dependent on the comparison result.

For optimizing the operation, in the inventive method and device or system a comparison of an executed sequence of processing or operating steps is made to data deposited in a data bank with reference to possible processing or operating sequences. Prompts about processing or operating steps meaningfully following the executed processing or operating steps are provided on the basis of the executed processing or operating steps. Further, it is possible to provide warnings if the executed sequence of processing or operating steps does not seem meaningful.

The medical device system has an operating device for the execution of the processing or operating steps. This can include switches, buttons, voice input, etc. A multitude of different means is also available for the output of the operating prompts. In particular, these can include acoustic means such as signal tones or voice output and optical means such as a picture screen or display.

In an embodiment of the invention parameter settings or functions of the medical device or system are automatically executed when a processing or operating sequence is recognized.

In accordance with the invention, specific sequence of processing or operating steps executed by a person, referred to as processing or operating sequences or “use cases”, are recognized, for example by comparison to the processing or operating sequences deposited in the data bank, and are logged. Advantageously, new sequences of processing or operating steps are also logged, i.e. sequences for which there are no corresponding processing or operating sequences deposited in the data bank. Particularly given these latter sequences of processing or operating steps, it is necessary to produce an evaluation in order to be able to allocate meaningful operating prompts, for example operating steps to be meaningfully executed following thereupon. For this purpose, a comparison of the new sequence of processing or operating steps to data of possible processing or operating sequences deposited in a further data bank, the master data bank, first ensues.

The data bank and the master data bank preferably are connected to one another via a computer network, for example the Internet. As a result, data of possible processing or operating sequences or a number of identical medical devices and systems can be centrally stored in the master data bank and can be correspondingly managed and evaluated there. Proceeding from the master data bank, the data bank directly allocated to the medical device or system can then be updated and expanded via the computer network. Given a sequence of processing or operating steps executed by the person for which there are no data in the data bank, a comparison to the data of the processing or operating sequences deposited in the master data bank first ensues. When a processing or operating sequence that has already been evaluated and agrees with the sequence of processing or operating steps is present, then these data are transmitted into the data bank locally allocated to the medical device or system, so that prompts about the operation of the medical device or system can be provided, or settings, and functions can be automatically executed given a repeat execution of the sequence of processing or operating steps.

The data transmission between the data banks can be manually triggered. Preferably, however, it ensues automatically, for example in conjunction with a time control.

When a sequence of processing or operating steps cannot be associated with either a processing or operating sequence of the data bank locally allocated to the medical device or system or a processing or operating sequence of the master data bank, then the invention advantageously provides for the use of expert knowledge. To this end, the sequence of processing or operating steps executed by the user and logged is made available to an expert via the computer network. The expert analyzes the sequence of processing or operating steps and can evaluate or optimize them. Operating prompts and, if needed, automatically executable parameter settings and functions are allocated to individual processing or operating steps to the evaluated processing or operating sequence that has thus arisen. Given a sequence of processing or operating steps, a prompt to possible, further meaningful processing or operating steps can ensue. The data with respect to the processing or operating sequence formed in this way are then deposited in the master data bank. By this means, a number of data banks of medical devices or systems connected to the master data bank via the network can then be updated and expanded. The newly evaluated processing or operating sequence then also is available to these devices and systems.

Of course, the expert—in conjunction with the master data bank—also can update or modify data of a processing or operating sequence in a data bank locally allocated to a medical device or system.

An advantage of the invention is avoiding operating errors. When an intended purpose of the user, i.e. an intended processing or operating sequence, can be unambiguously recognized as a result of a specific sequence of processing or operating steps entered by the user, then operating steps that meaningfully follow an executed operating step can be presented, settings of parameters and functions can be automatically undertaken or specific functions of the system can be blocked. Operating errors thus can be prevented.

By outputting operating prompts for a user, or by the automatic execution of functions, the invention also contributes to enhancing throughput using the medical device or system.

Another advantage of the invention is the service concept whereby an expert who processes processing or operating sequences of the medical device or system is relieved of much routine work, since only new sequences of processing or operating steps are submitted to the expert for processing and evaluation. As a result, the expert can concentrate on the demanding and difficult special cases for which the expert is truly needed. Processing or operating sequences that already have been evaluated and processed and stored in the master data bank can be automatically communicated automatically to the user of a medical device or system.

### DESCRIPTION OF THE DRAWING

The single FIGURE is a schematic block diagram of a medical apparatus constructed and operating in accordance with the principles of the present invention.

### DESCRIPTION OF THE PREFERRED EMBODIMENTS

The FIGURE schematically shows a software-controlled medical device or system 1 such as, for example, a hearing aid device in combination with a programming system or a computed tomography device. The medical device or system 1 has an input/output unit 2 by means of which a user 3 can operate the medical device or system 1. The input/output unit 2, for example, can include a keyboard and a monitor. Via the input/output unit 2, the user 3 executes a sequence of processing or operating steps for the operation of the medical device or system 1. As used in the context of the invention “operating” encompasses the control, programming, setting, maintenance, adaptation as well as monitoring of the medical device or system 1. The sequence of processing or operating steps executed by the user 3 via the input/output unit 2 is monitored by an operating software allocated to the medical device or system in combination with a processor (not shown). This operating software attempts to recognize the intended application (use case) of the user 3 and to optimize this application. For recognizing the intent of the user 3, a comparison of the processing or operating steps executed by the user 3 to data with respect to processing or operating sequences deposited in a data bank 4 is carried out. When the executed sequence of processing or operating steps agrees with a part of a processing or operating sequence stored in the data bank 4, then the user 3 can be provided—via the input/output unit—with prompts with respect to meaningful, further processing or operating steps. These prompts can likewise be stored in the data bank 4, linked to the corresponding processing or operating sequence, and can be converted into operating instructions perceptible to the user 3 by the processor, the operating software and corresponding output unit 2.

When the operating software recognizes an application intended by the user 3, i.e. an intended processing or operating sequence, it is also possible to automatically implement settings and functions of the medical device or system 1. On the basis of the data that relate to this processing or operating sequence and that are stored in the data bank 4, the processor and the operating software generate and execute corresponding commands for this purpose. Particularly given more complex applications of the medical device or system 1, this can contribute to significantly facilitating and simplifying the operation.

If the sequence of processing or operating steps executed by the user 3 cannot be unambiguously allocated to a processing or operating sequence stored in the data bank 4, then the medical device or system 1 sets up a connection to a computer 6 via the Internet 5, said computer 6 being in turn connected to a master data bank 7. The medical device or system 1 as well as the computer 6 have the required (known) connection means for this purpose. The contacting of the computer unit 6 can be automatically or manually initiated. In particular, it is expedient to first log a sequence of processing or operating steps executed by the user 3 that does not have an equivalent with a processing or operating sequence stored in the data bank 4, before contact with the computer unit 6 is set up. The data with respect to this new processing or operating sequence are then compared to data of the processing or operating sequences stored in the master data bank 7. When the processing or operating sequence executed by the user 3 is contained in the master data bank 7, then an updating of the data bank 4 ensues with the data with respect to the new processing or operating sequence from the master data bank 7.

If the processing or operating sequence executed by the user 3 has no equivalent either in the data bank 4 or in the master data bank, then processing and evaluation of this processing or operating sequence is carried out by an expert 8. The latter uses a computer unit 9 for this purpose in which expert software is executed for the analysis of the processing or operating steps executed by the user 3. The expert 8 processes, checks and evaluates the executed processing or operating sequence and, in particular, determines whether this

a) is rational,

b) is irrational since, for example, there is a more reasonable or effective equivalent, or

c) is faulty since, for instance, an operation in the indicated way is not intended.

Further, the individual steps of the processing or operating sequence are provided with additional information that provide the user 3 with prompts to meaningful steps following a specific processing or operating step during the implementation of the processing or operating sequence. This information additionally or alternatively can be used to automatically implement settings and functions of the medical device or system 1. The data of the new processing or operating sequence processed in this way are then deposited in the master data bank 7 and possibly in the data bank 4 as well. The usage frequency of the processing or operating sequences that are executed with a medical device or system 1 and that are stored in the data bank 4 are logged.

The software for the control of the medical device or system 1 can now access the updated data with respect to the processing or operating steps during ongoing operations and, given recognition of an intended processing or operating sequence that is present in the data bank 4, can give prompts about the optimization of the executive sequences or can automatically implement settings and functions. The software also can optimize executive sequences on its own.

in accordance with the invention a combination of additional software functionality, networking of the applicable computers and the performance of a service that is used to update the appertaining data banks and to analyze and optimize the operation of the medical device.

Specifically, the medical device or system 1 can be implemented as a system with a hearing aid device and a programming device connected thereto in which adaptation software is implemented. The devices are becoming increasingly complex, and the users (hearing aid acousticians) are being increasingly overtaxed. This is because the acoustician naturally wants to and must demonstrate know-how about the adaptation of the hearing aids to a hearing-impaired person by demonstrating an awareness of all functions of the software and of the hearing aid devices, but the hearing aid devices offer an unsurveyable multitude of possibilities. An acceptable but not optimum setting of a hearing aid device thus often occurs. The invention can be of assistance by the adaptation software being supplied together with a data bank 4 in which the data with respect to possible processing or operating sequences of the software and of the hearing aid are stored. The data bank 4 contains not only the data of meaningful processing or operating sequences but also data with respect to processing or operating sequences that are not meaningful, so that these can also be recognized and alternatives can be presented. A possible operating sequence can conclude the following operating steps:

a) import of a loudness scaling characteristic,

b) readout of the connected hearing aid device at the right ear, and

c) implementation of a presentation with selection of an audio characteristic without loudness balancing and with a loudness scaling.

An operating step that is not meaningful could thereby be the direct input of loudness scaling data. If the user 3 makes an effort to execute such an operating step, then the user is automatically provided with a warning as well as with meaningful alternatives, for example by means of an acoustic alarm signal as well as a text output on a monitor with meaningful operating steps. Clicking on a displayed loudness diagram can also automatically effect an algorithm for the import of loudness data. This, however, is only possible when corresponding commands of the processing and operating sequence are allocated as data and deposited in the data bank 4.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for operating a medical apparatus comprising the steps of:
for a medical apparatus, storing in a databank a plurality of possible execution sequences, selected from the group consisting of processing steps and operating steps, for said medical apparatus;
operating said medical apparatus using a manually-entered execution sequence and, as said manually-entered execution sequence is executed by said medical apparatus, comparing said manually-entered execution sequence to said plurality of execution sequences in said databank and obtaining a comparison result; and
optimizing operation of said medical apparatus dependent on said comparison result.

2. A method as claimed in claim 1 wherein the step of optimizing operation of said medical apparatus comprises automatically providing humanly perceptible operating prompts.

3. A method as claimed in claim 1 wherein the step of optimizing operation of said medical apparatus comprises automatically altering said manually-entered execution program using alterations, selected from the group consisting of parameter settings and functions, derived from said comparison result.

4. A method as claimed in claim 1 comprising automatically logging steps in said manually-entered execution sequence.

5. A method as claimed in claim 1 comprising the additional step of evaluating said manually-entered execution sequence.

6. A method as claimed in claim 5 wherein the step of evaluating said manually-entered execution sequence comprises evaluating said manually-entered execution sequence in a master databank separate from said databank.

7. A method as claimed in claim 5 wherein the step of evaluating said manually-entered execution sequence comprises transmitting said manually-entered execution sequence to a location of an expert, and evaluating said manually-entered execution sequence at said location by said expert.

8. A method as claimed in claim 5 wherein the step of evaluating said manually-entered execution sequence comprises transferring said manually-entered execution sequence to an addressee of a computer network, and evaluating said manually-entered execution sequence at said addressee,

9. A method as claimed in claim 8 comprising automatically transferring said manually-entered execution sequence to said addressee.

10. A method as claimed in claim 8 comprising employing the Internet as said computer network.

11. A method as claimed in claim 5 wherein the step of evaluating said manually-entered execution sequence produces an evaluation result, and comprising the additional step of storing said evaluation result in a master databank separate from said databank.

12. A method as claimed in claim 5 wherein the step of evaluating said manually-entered execution sequence produces an evaluation result, and comprising the additional step of storing said evaluation result in said databank as a further execution sequence in said plurality of possible execution sequences.

13. A system comprising:
a medical apparatus;
a databank in which a plurality of possible execution sequences for said medical apparatus are stored, selected from the group consisting of processing steps and operating steps;
a control unit for operating said medical apparatus, said control unit having access to said data bank;
an input/output unit allowing entry by a user, into said control unit, of a manually-entered execution sequence for said medical apparatus; and
said control unit operating said medical apparatus using said manually-entered execution sequence and, as said manually-entered execution sequence is executed by said medical apparatus, said control unit comparing said manually-entered execution sequence to said plurality of execution sequences in said databank and obtaining a comparison result, and optimizing operation of said medical apparatus dependent on said comparison result.

14. A system as claimed in claim 13 wherein said control unit optimizes operation of said medical apparatus by automatically providing humanly perceptible operating prompts at said input/output unit.

15. A system as claimed in claim 13 wherein said control unit optimizes operation of said medical apparatus by automatically altering said manually-entered execution program using alterations, selected from the group consisting of parameter settings and functions, derived from said comparison result.

16. A system as claimed in claim 13 said control unit automatically logs steps in said manually-entered execution sequence.

17. A system as claimed in claim 13 comprising a master databank separate from said databank for evaluating said manually-entered execution sequence comprises evaluating said manually-entered execution sequence.

18. A system as claimed in claim 17 wherein said mater databank produces an evaluation result from evaluating said manually-entered execution sequence stores said evaluation result.

19. A system as claimed in claim 18 wherein said control unit stores said evaluation result in said databank as a further execution sequence in said plurality of possible execution sequences.

20. A system as claimed in claim 13 wherein said control unit transmits said manually-entered execution sequence to a location of an expert for evaluating said manually-entered execution sequence.

21. A system as claimed in claim 20 wherein said expert produces an evaluation result, and transmits said evaluation result to said control unit, and wherein said control unit stores said evaluation result in said databank as further execution sequence in said plurality of possible execution sequences.

22. A system as claimed in claim 13 wherein said control unit transfers said manually-entered execution sequence to an addressee of a computer network for evaluating said manually-entered execution sequence at said addressee,

23. A system as claimed in claim 22 wherein evaluation of said manually-entered execution sequence at said addressee produces an evaluation result, and wherein said evaluation result is transferred via said computer network to said control unit, and wherein said control unit stores said evaluation result in said databank as a further execution sequence in said plurality of possible execution sequences

24. A system as claimed in claim 22 wherein said control unit automatically transfers said manually-entered execution sequence to said addressee.

25. A system as claimed in claim 22 wherein said control unit employs the Internet as said computer network.
